(19) United States
(12) Patent Application Publication
(10) Pub. No.: US 2024/0317675 A1
(43) Pub. Date: Sep. 26, 2024

(54) OXIDIZABLE COMPOUND, ELECTROCHROMIC COMPOSITION, AND ELECTROCHROMIC DEVICE

(71) Applicant: INDUSTRIAL TECHNOLOGY RESEARCH INSTITUTE, Hsinchu (TW)

(72) Inventors: Guey-Sheng LIOU, Puli Township (TW); Chyi-Ming LEU, Jhudong Township (TW); Jheng-Ying LI, Taoyuan City (TW); Hsiang-Jui CHEN, Hsinchu City (TW); Yu Jen SHAO, Taipei (TW); Min Hsiu TU, Taipei (TW); Hou Lin LI, Taipei (TW)

(73) Assignee: INDUSTRIAL TECHNOLOGY RESEARCH INSTITUTE, Hsinchu (TW)

(21) Appl. No.: 18/395,277

(22) Filed: Dec. 22, 2023

Related U.S. Application Data
(60) Provisional application No. 63/491,807, filed on Mar. 23, 2023.

(30) Foreign Application Priority Data
Oct. 31, 2023 (TW) 112141713

(51) Int. Cl. C07C 217/92; C09K 9/02; G02F 1/1514; G02F 1/155

(52) U.S. Cl. CPC C07C 217/92; C09K 9/02; G02F 1/1514; G02F 1/155; C09K 2211/1007; C09K 2211/1014

(57) ABSTRACT

An electrochromic composition includes an oxidizable compound, a reducible compound, and a solvent. The oxidizable compound includes

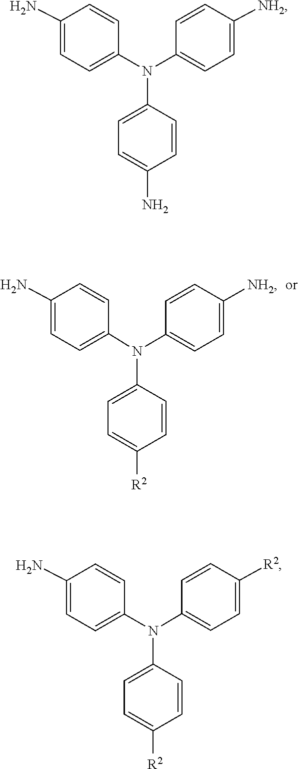

wherein each R² is independently —N(R¹)², —OR¹, or —R¹, and R¹ is C₁₋₃ alkyl group. The reducible compound has a chemical structure of

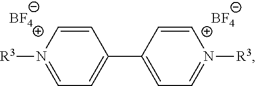

and each R³ is independently C₄₋₁₂ alkyl group, —OCₙH₂ₙ₊₁,

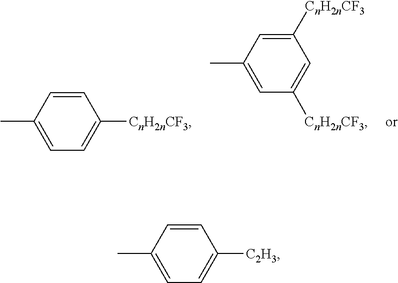

and n=0˜7.

### CROSS REFERENCE TO RELATED APPLICATIONS

The present application claims the benefit of U.S. Provisional Application No. 63/491,807 filed on Mar. 23, 2023, and claims priority from Taiwan Application Serial Number 112141713, filed on Oct. 31, 2023, the disclosure of which are hereby incorporated by reference herein in its entirety.

### TECHNICAL FIELD

The technical field relates to an oxidizable compound, and relates to an electrochromic composition including the same and an electrochromic device utilizing the same.

### BACKGROUND

Electrochromic related products are attractive in green energy industries due to their low driving voltage (<3.0 V) and bistable properties. This technology is regarded as an important industry in the next decades, in which electrochromic materials will play a critical role. Major electrochromic materials are inorganic oxides. The inorganic oxide has shortcomings such as a slow electrochromic rate and less color variation. In organic systems, the electrochromic conjugated polymer has more color variation and fast electrochromic rates. The electrochromic conjugated polymer in a neutral state has an appearance of a deep color due to its conjugated length. Although the deep color can be lightened by applying voltage, the conjugated polymer cannot be fully transparent. In manual dynamic control selections, the electrochromic conjugated polymer must be electrified to enter a transparent state, thereby leading to high energy consumption. A general electrochromic composition containing a small organic molecule has an overly high driving voltage, thereby limiting the application of the small organic molecule in the electrochromic field. Accordingly, for application in electrochromic related products, a novel electrochromic material is called for.

### SUMMARY

One embodiment of the disclosure provides an oxidizable compound, having a chemical structure of

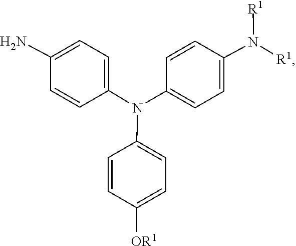

wherein each R¹ is independently C₁₋₃ alkyl group.

One embodiment of the disclosure provides an electrochromic composition, including: an oxidizable compound; a reducible compound; and a solvent. The oxidizable compound includes

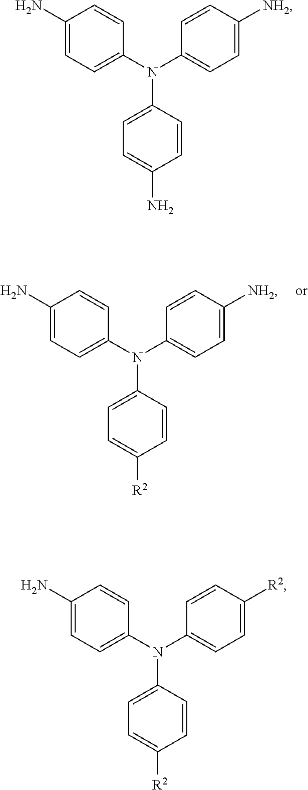

wherein each R² is independently —N(R¹)₂, —OR¹, or —R¹, and R¹ is C₁₋₃ alkyl group. The reducible compound has a chemical structure of

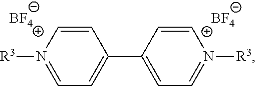

and each R³ is independently C₄₋₁₂ alkyl group, —OCₙH₂ₙ₊₁,

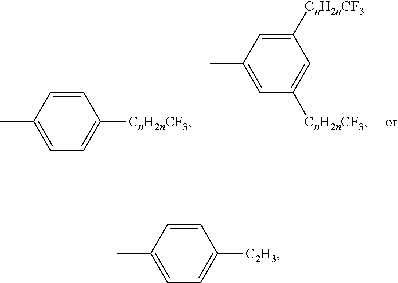

and n=0˜7.

One embodiment of the disclosure provides an electrochromic device, including a first transparent conductive layer, a second transparent conductive layer; and the described electrochromic composition disposed between the first transparent conductive layer and the second transparent conductive layer.

A detailed description is given in the following embodiments with reference to the accompanying drawings.

### BRIEF DESCRIPTION OF THE DRAWINGS

The disclosure can be more fully understood by reading the subsequent detailed description and examples with references made to the accompanying drawings, wherein:

FIGS. 1A to lE are the cyclic voltammetry (CV) spectra of the electrochromic compositions in some embodiments of the disclosure.

FIGS. 2A to 2D are absorption spectra of the electrochromic compositions under different driving voltages in some embodiments of the disclosure.

FIGS. 3A to 3E are CV spectra of solutions of the triphenyl amine molecules and the ammonium salt in some embodiments of the disclosure.

FIGS. 4A to 4D are CV spectra of the triphenyl amine molecules and the ammonium salt after several cycles in some embodiments of the disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

One embodiment of the disclosure provides an oxidizable compound, having a chemical structure of

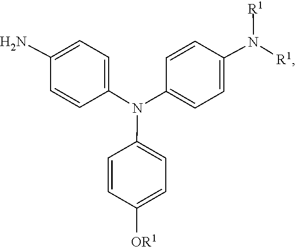

wherein each R¹ is independently C₁₋₃ alkyl group. For example, the oxidizable compound has a chemical structure of

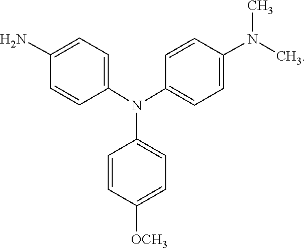 [SMILES: COc1ccc(N(c2ccc(N)cc2)c2ccc(N(C)C)cc2)cc1]

In some embodiments, the oxidizable compound can be synthesized by coupling 4-bromophenyl alkyl ether and 4-dialkylamino-4′-nitro diphenylamine to form a nitro triphenyl amine compound. It should be understood that the three R¹s in the below Formula can be the same or different from each other as necessary.

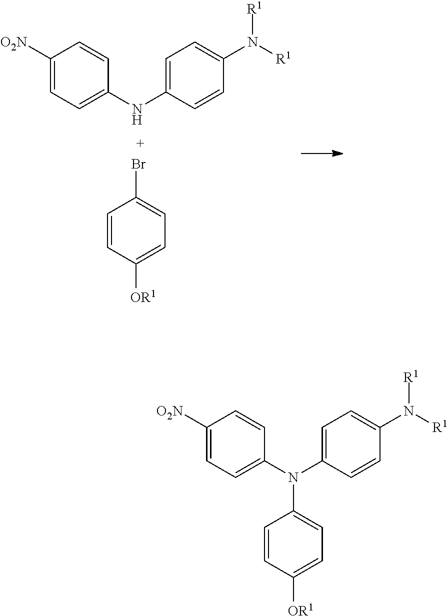

Subsequently, the nitro group of the nitro triphenyl amine is reduced to form amino triphenyl amine, as shown in below Formula.

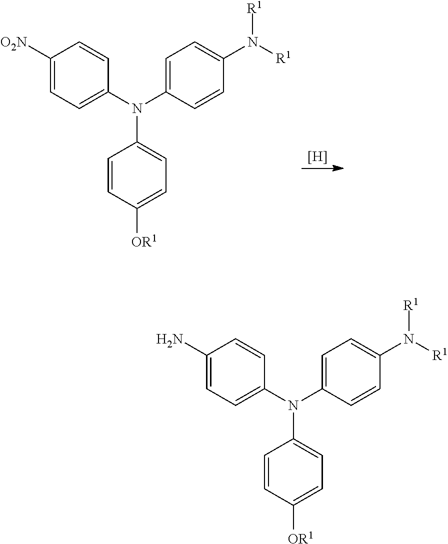

The reaction is used to illustrate a possible path to synthesize the oxidizable compound such as triphenyl amine, which is not the only applicable synthesis path. One skilled in the art may adopt any applicable synthesis way as necessary to form the disclosed oxidizable compound in the disclosure.

One embodiment of the disclosure provides an electrochromic composition, including: an oxidizable compound; a reducible compound; and a solvent. The oxidizable compound includes

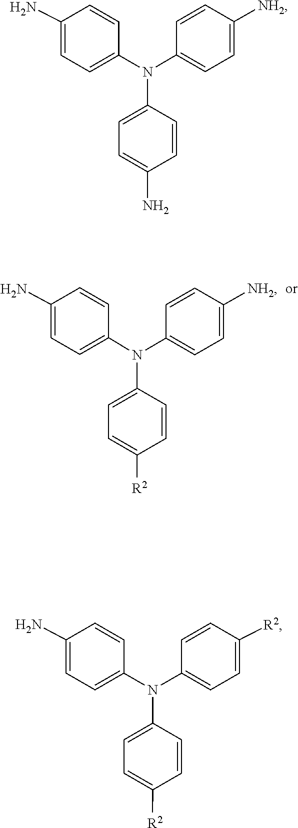

wherein each R² is independently —N(R¹)₂, —OR¹, or —R¹, and R¹ is C₁₋₃ alkyl group. The reducible compound has a chemical structure of

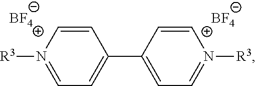

and each R³ is independently C₄₋₁₂ alkyl group, —OCₙH₂ₙ₊₁,

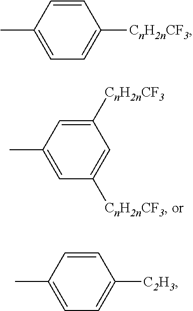

and n=0˜7.

In some embodiments, the oxidizable compound is

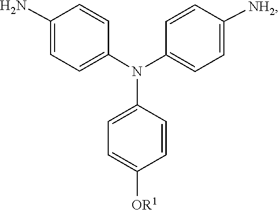

such as

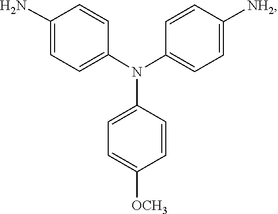 [SMILES: COc1ccc(N(c2ccc(N)cc2)c2ccc(N)cc2)cc1]

In some embodiments, the oxidizable compound is

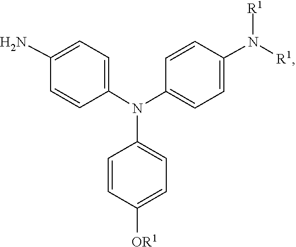

such as

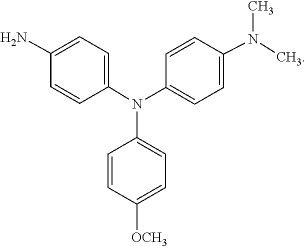 [SMILES: COc1ccc(N(c2ccc(N)cc2)c2ccc(N(C)C)cc2)cc1]

In some embodiments, the oxidizable compound is

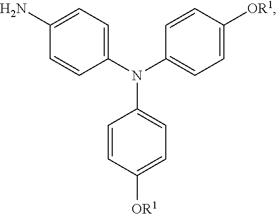

such as

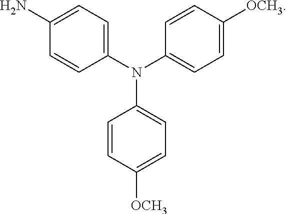 [SMILES: COc1ccc(N(c2ccc(N)cc2)c2ccc(OC)cc2)cc1]

In some embodiments, the solvent includes propylene carbonate, γ-butyrolactone, acetonitrile, propionitrile, glutaronitrile, methylglutaronitrile, 3,3′-oxydipropionitrile, hydroxypropionitrile, dimethylformamide, N-methyl tetrahydropyrrolidone, sulfolane, 3-methylsulfolane, or a combination thereof.

In some embodiments, the electrochromic composition further includes an electrolyte such as an ammonium salt. For example, the ammonium salt can be TBABF4 ((N(C₄H₉)₄)⁺BF₄⁻), TBAP ((N(C₄H₉)₄⁺ClO₄⁻), another suitable ammonium salt, or a combination thereof. The electrolyte is beneficial to balance charge and transfer current.

In the electrochromic composition, the oxidation potential of the oxidizable compound matches the reduction potential of the reducible compound, such as viologen (e.g. the potential difference therebetween is less than 0.15 V), thereby improving the cycling stability and lowering the driving voltage.

In some embodiments, the concentration of the oxidizable compound and the concentration of the reducible compound in the electrochromic composition can be independently 0.003 M (mole/L) to 0.1 M, preferably 0.005 M to 0.05 M, more preferably of 0.01 M to 0.025 M, and most preferably 0.012 M to 0.0.18 M.

In some embodiments, the oxidizable compound and the reducible compound in the electrochromic composition may have a molar ratio of 7:3 to 3:7, preferably 6:4 to 4:6, and most preferably 5:5.

One embodiment of the disclosure provides an electrochromic device, including a first transparent conductive layer, a second transparent conductive layer; and the described electrochromic composition disposed between the first transparent conductive layer and the second transparent conductive layer. In some embodiments, the first transparent conductive layer and the second transparent conductive layer include indium tin oxide, indium zinc oxide, aluminum zinc oxide, cadmium tin oxide, tin oxide, or zinc oxide. In general, the first transparent conductive layer can be formed on a first transparent substrate, and the second transparent conductive layer can be formed on a second transparent substrate. The first transparent substrate and the second transparent substrate are assembled, and their edges are adhered by a gap glue to define the space between the first transparent substrate and the second transparent substrate. The electrochromic composition is injected into the space through a hole in the gap glue, and the hole is then sealed to complete the electrochromic device. It should be understood that the above method of forming the electrochromic device is only for illustration rather than limiting the disclosure thereto. One skilled in the art may adopt any applicable way to put the electrochromic composition between the first transparent conductive layer and the second transparent conductive layer to form the so-called electrochromic device. The electrochromic device is transparent when not powered. When a positive voltage is applied to the electrochromic device, the color of the electrochromic composition is gradually deeper. Once the power is off, the electrochromic composition will return to the original transparent state in a short time (<1 second).

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

### EXAMPLES

In following Examples, the triphenyl amine TAP-1N

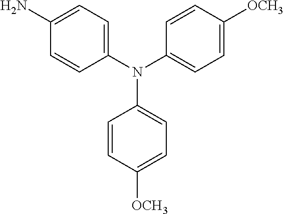 [SMILES: COc1ccc(N(c2ccc(N)cc2)c2ccc(OC)cc2)cc1]

was prepared according to Macromolecules, 2008, 41 (5), 1667-1674, the triphenyl amine TPA-2N

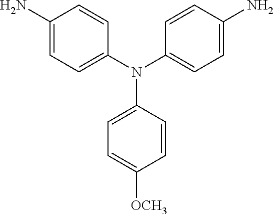 [SMILES: COc1ccc(N(c2ccc(N)cc2)c2ccc(N)cc2)cc1]

was prepared according to Journal of Materials Chemistry, 2007, 17 (10), 1007-1015, the triphenyl amine TPA-3N

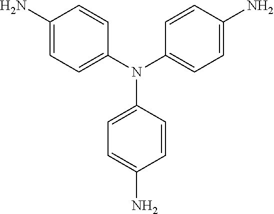 [SMILES: Nc1ccc(N(c2ccc(N)cc2)c2ccc(N)cc2)cc1]

was prepared according to Journal of Materials Chemistry C, 2018, 6 (48), 13345-13351, and triphenyl amine 3PiAB

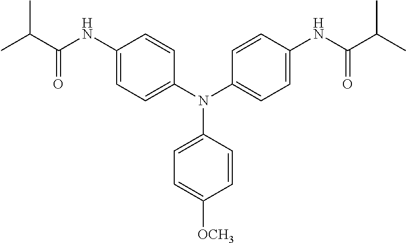 [SMILES: COc1ccc(N(c2ccc(NC(=O)C(C)C)cc2)c2ccc(NC(=O)C(C)C)cc2)cc1]

was prepared according to Taiwan Patent No. TWI551574. The ammonium salt TBABF4 (N(C₄H₉)₄)⁺BF₄⁻) was commercially available from Uni-Onward Co., and the ammonium salt TBAP (N(C₄H₉)₄⁺ClO₄⁻) was commercially available from Uni-Onward Co.

### Synthesis Example 1 (Synthesis of Heptyl Viologen HV)

4,4′-bipyridine (1.56 g, 0.01 mole) and 1-bromoheptane (3.58 g, 0.02 mole) were dissolved in solvent of 60 g of water and 40 g of ethanol, and reacted at room temperature for 2 hours in the presence of acetonitrile (0.01 mole) serving as catalyst, and excess amount of sodium fluoroborate (2.19 g, 2 mole) was then added to the reaction to perform ion-exchange. The mixture was then stirred for 1 hour, and ethanol was then removed. The crude was precipitated by ice-bath, and then filtered by a filter paper with a pore size of 1 μm to collect white crystal. The white crystal was dried in an oven at 80° C. to obtain heptyl viologen HV, which had a hydrogen spectrum as below: ¹H NMR (400 MHz, D₂O): δ 9.02 (d, J=5.2 Hz, 4H, viologen), 8.44 (d, J=5.2 Hz, 4H, viologen), 4.59 (t, J=6.0 Hz, 4H, —CH₂—), 1.93 (m, 4H, —CH₂—), 1.18 (m, 12H, —CH₂—), 0.79 (t, J=6.4 Hz, 6H, —CH₃). The heptyl viologen HV had a chemical structure of

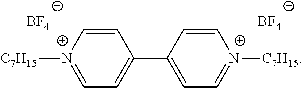

### Synthesis Example 2 (Synthesis of TPA-ADM)

4-Bromoanisole (2.24 g, 1.51 mL, 12.00 mmole) and 4-dimethylamino-4′-nitro diphenylamine (2.57 g, 10.00 mmole, synthesized according to China Patent No. CN105924361B) were dissolved in 30 mL of 1,3-dichlorobenzen under nitrogen. Copper powder (0.76 g, 12.00 mole), potassium carbonate (3.32 g, 24.00 mmole) and 18-crown-6 ether (0.26 g, 1.00 mmole) were added to the above solution. The reaction mixture was heated to 160° C. and then stirred for 2 days, and then filtered while hot by Celite® to remove the residual copper powder and salts. The filtrate was cooled and then poured into 200 mL of water, and then extracted by 100 mL of dichloromethane 3 times to obtain an organic layer. The organic layer was concentrated using a rotatory pump to remove solvent and obtain crude. The crude was purified by chromatography to obtain a red crystal product TPA-NDM (0.78 g, yield=21%). The chromatography was performed with a silica gel column and an eluent of chloroform/hexane (1/1). TPA-NDM had a melting point of 108° C. to 111° C. TPA-NDM had a hydrogen spectrum as below: ¹H NMR (600 MHz, DMSO-d₆) δ (ppm): 7.99 (d, J=9.4 Hz, 2H), 7.23 (d, J=9.0 Hz, 2H), 7.12 (d, J=8.9 Hz, 2H), 7.00 (d, J=8.8 Hz, 2H), 6.76 (d, J=9.0 Hz, 2H), 6.61 (d, J=9.4 Hz, 2H), 3.77 (s, 3H), 2.91 (s, 6H). TPA-NDM had a carbon spectrum as below: ¹³C NMR (150 MHz, DMSO-d₆) δ (ppm): 157.44, 154.39, 148.92, 137.67, 137.36, 133.28, 128.33, 128.02, 125.65, 115.28, 114.10, 113.36, 55.30, 40.06. The reaction is shown below:

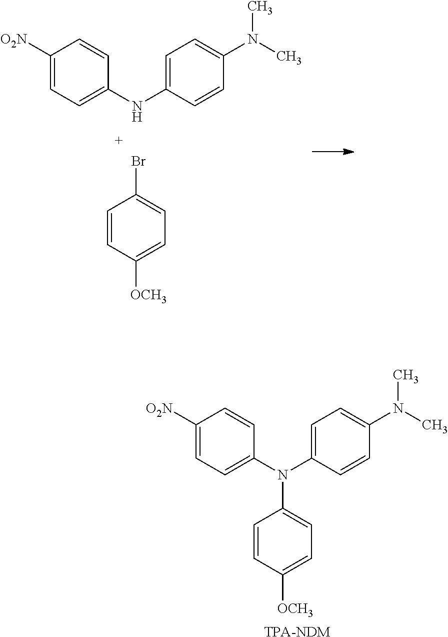

TPA-NDM (0.73 g, 2.00 mole) was dissolved in 15 mL of ethanol under nitrogen, and Pd/C (10% Pd, 0.036 g) was added to the solution. The reaction mixture was heated to reflux, and hydrazine hydrate (1.00 mL, 20 mmole) was dropwise added into the reaction mixture. After reacting and being refluxed for 12 hours, the reacted mixture was filtered while hot by Celite® to remove Pd/C. The filtrate was cooled and stood for 1 day to obtain crystal such as white solid TPA-ADM (0.60 g, yield=91%). The TPA-ADM had a melting point of 97° C. to 99° C. TPA-ADM had a hydrogen spectrum as below: ¹H NMR (600 MHz, DMSO-d₆) δ (ppm): 6.80 (d, J=9.0 Hz, 2H), 6.77-6.71 (m, 4H), 6.70 (d, J=8.6 Hz, 2H), 6.64 (d, J=9.0 Hz, 2H), 6.50 (d, J=8.6 Hz, 2H), 4.85 (s, 2H), 3.67 (s, 3H), 2.82 (s, 6H). TPAD-ADM had a carbon spectrum as below: ¹³C NMR (150 MHz, DMSO-d₆) δ (ppm): 153.20, 146.16, 144.51, 142.53, 138.36, 137.16, 125.81, 124.36, 121.88, 114.79, 114.33, 113.65, 55.15, 40.59. The reaction is shown below:

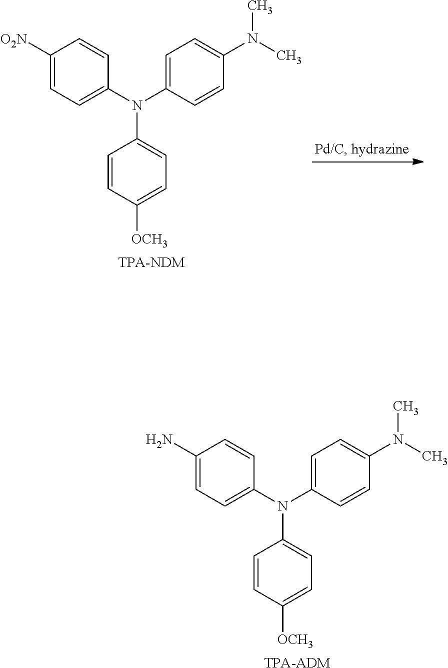

### Example 1

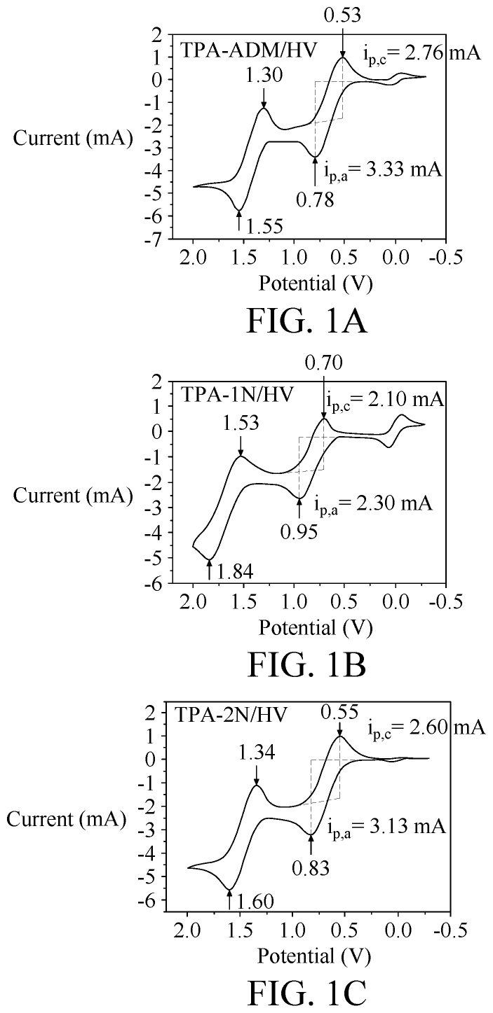

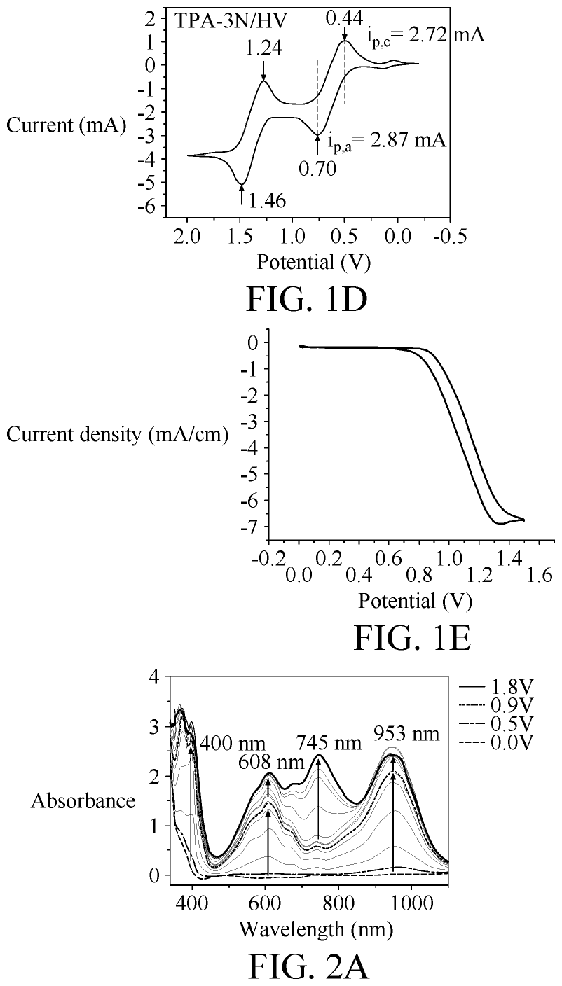

An electrochromic composition was prepared, which contained solvent of γ-butyrolactone (0.048 mL), 0.72 μmole of the triphenyl amine compound TPA-ADM (0.015 M), 0.72 μmole of the heptyl viologen HV (0.015 M), and 4.8 μmole of TBABF4 (0.1 M). The electrochromic composition was interposed between two ITO glasses (2 cm*2 cm), and analyzed by electrochemical analytical instrument CHI-6122E (commercially available from CH instruments) to measure the cyclic voltammetry (CV) spectrum of the electrochromic composition (scan rate was 50 mV/s). The CV spectrum of the electrochromic composition is shown in FIG. 1A.

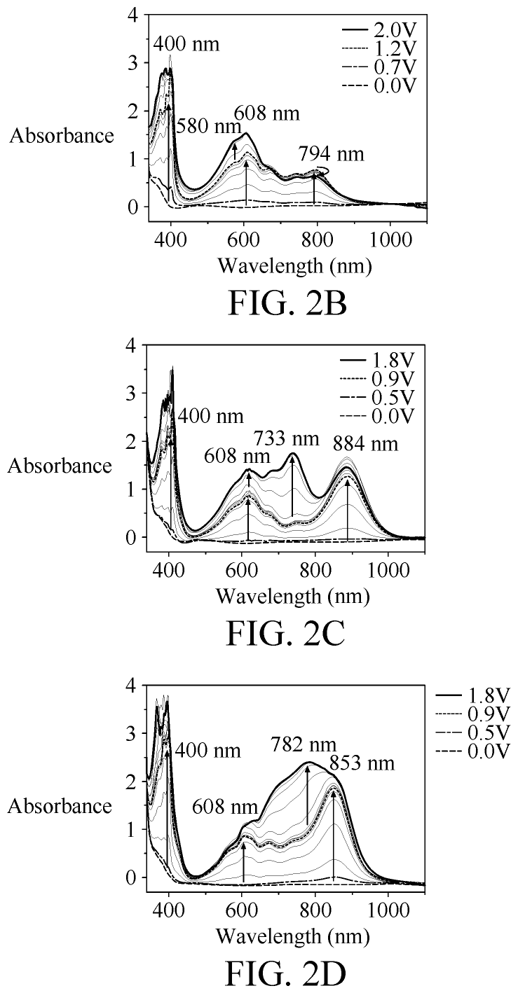

In addition, different driving voltages were applied to the electrochromic composition to measure its absorption spectra, as shown in FIG. 2A. As known from FIG. 2A, when the driving voltage achieved 0.9 V, the electrochromic composition would have an electrochromic effect.

When no driving voltage was applied, the electrochromic composition was colorless (L*=97.87, a*=−1.08, b*=6.72). When a driving voltage of 0.9 V was applied, the electrochromic composition changed to deep sky blue (L*=53.50, a*=−26.93, b*=−37.61). When a driving voltage of 1.2 V was applied, the electrochromic composition changed to grey blue (L*=47.57, a*=−34.86, b*=−35.81). For a definition of L*, a*, and b*, refer to the CIELAB color space. In general, a lower L* value means a deeper color. The driving voltages and the color variation effect of the electrochromic composition are tabulated in Table 1.

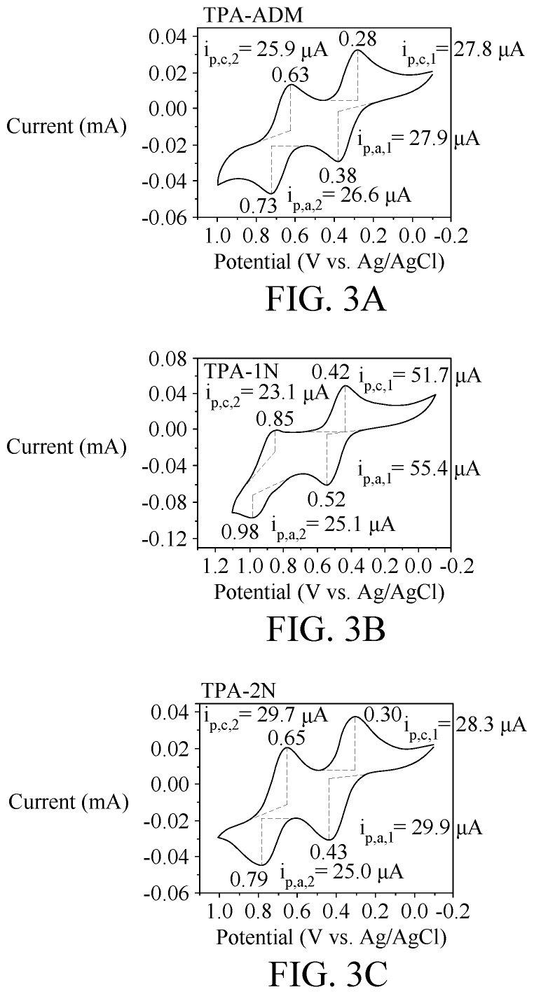

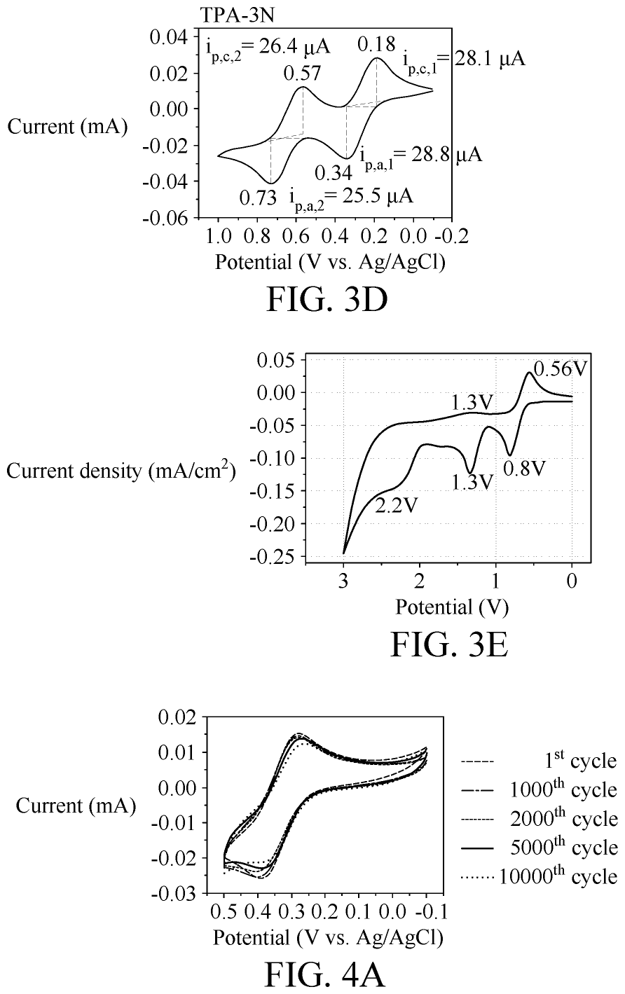

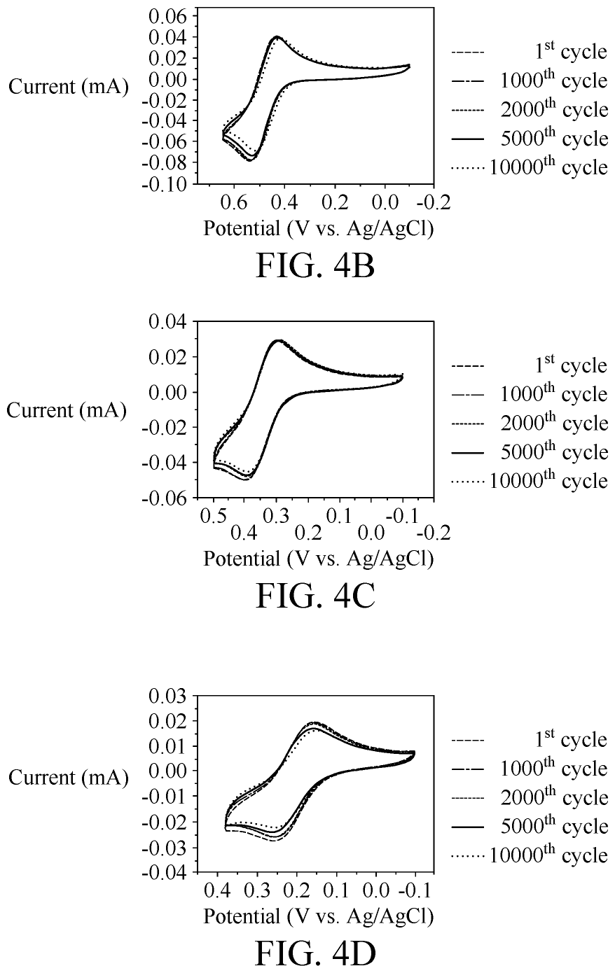

0.5 μmole of TPA-ADM was dissolved in 1 mL of γ-butyrolactone solution of TBAP (1M), and analyzed by a three-electrode system to measure the CV spectrum of TPA-ADM, as shown in FIG. 3A. A platinum mesh (1 cm*2.5 cm) served as a working electrode, a platinum wire served as an auxiliary electrode, and Ag/AgCl served as a reference electrode. The CV spectra of the TPA-ADM solution after different scan cycles (scan rate was 100 mV/s) are shown in FIG. 4A. The oxidation-reduction potentials of TPA-ADM could be known from the CV spectra. The absorption residual value (of maximum UV absorption band) after 10000 cycles from 0.5 V to −0.1 V is tabulated in Table 2.

### Example 2

An electrochromic composition was prepared, which contained solvent of γ-butyrolactone (0.048 mL), 0.72 μmole of the triphenyl amine compound TPA-1N (0.015 M), 0.72 μmole of the heptyl viologen HV (0.015 M), and 4.8 μmole of TBABF4 (0.1 M). The electrochromic composition was interposed between two ITO glasses (2 cm*2 cm), and analyzed by electrochemical analytical instrument CHI-6122E (commercially available from CH instruments) to measure the cyclic voltammetry (CV) spectrum of the electrochromic composition (scan rate was 50 mV/s). The CV spectrum of the electrochromic composition is shown in FIG. 1B.

In addition, different driving voltages were applied to the electrochromic composition to measure its absorption spectra, as shown in FIG. 2B. As known from FIG. 2B, when the driving voltage achieved 1.2 V, the electrochromic composition would have an electrochromic effect. When no driving voltage was applied, the electrochromic composition was colorless (L*=97.52, a*=−0.20, b*=4.67). When a driving voltage of 1.2 V was applied, the electrochromic composition changed to navy blue (L*=47.90, a*=5.66, b*=−55.12). When a driving voltage of 2.0 V was applied, the electrochromic composition changed to a deeper color (L*=31.09, a*=2.15, b*=−44.40). The driving voltages and the color variation effect of the electrochromic composition are tabulated in Table 1.

0.5 μmole of TPA-1 N was dissolved in 1 mL of γ-butyrolactone solution of TBAP (1M), and analyzed by a three-electrode system to measure the CV spectrum of TPA-1 N, as shown in FIG. 3B. The CV spectra of the TPA-1 N solution after different scan cycles (scan rate was 100 mV/s) are shown in FIG. 4B. The oxidation-reduction potentials of TPA-1N could be known from the CV spectra. The absorption residual value (of maximum UV absorption band) after 10000 cycles from 0.5 V to −0.1 V is tabulated in Table 2.

### Example 3

An electrochromic composition was prepared, which contained solvent of γ-butyrolactone (0.048 mL), 0.72 μmole of the triphenyl amine compound TPA-2N (0.015 M), 0.72 μmole of the heptyl viologen HV (0.015 M), and 4.8 μmole of TBABF4 (0.1 M). The electrochromic composition was interposed between two ITO glasses (2 cm*2 cm), and analyzed by electrochemical analytical instrument CHI-6122E (commercially available from CH instruments) to measure the cyclic voltammetry (CV) spectrum of the electrochromic composition (scan rate was 50 mV/s). The CV spectrum of the electrochromic composition is shown in FIG. 1C.

In addition, different driving voltages were applied to the electrochromic composition to measure its absorption spectra, as shown in FIG. 2C. As known from FIG. 2C, when the driving voltage achieved 0.9 V, the electrochromic composition would have an electrochromic effect. When no driving voltage was applied, the electrochromic composition was colorless (L*=98.23, a*=1.26, b*=2.69). When a driving voltage of 0.9 V was applied, the electrochromic composition changed to deep blue (L*=69.29, a*=−6.76, b*=−37.93). When a driving voltage of 1.8 V was applied, the electrochromic composition changed to deeper blue (L*=61.21, a*=−9.44, b*=−26.39). The driving voltages and the color variation effect of the electrochromic composition are tabulated in Table 1.

0.5 μmole of TPA-2N was dissolved in 1 mL of γ-butyrolactone solution of TBAP (1M), and analyzed by a three-electrode system to measure the CV spectrum of TPA-2N, as shown in FIG. 3C. The CV spectra of the TPA-2N solution after different scan cycles (scan rate was 100 mV/s) are shown in FIG. 4C. The oxidation-reduction potentials of TPA-2N could be known from the CV spectra. The absorption residual value (of maximum UV absorption band) after 10000 cycles from 0.5 V to −0.1 V is tabulated in Table 2.

### Example 4

An electrochromic composition was prepared, which contained solvent of γ-butyrolactone (0.048 mL), 0.72 μmole of the triphenyl amine compound TPA-3N (0.015 M), 0.72 μmole of the heptyl viologen HV (0.015 M), and 4.8 μmole of TBABF4 (0.1 M). The electrochromic composition was interposed between two ITO glasses (2 cm*2 cm), and analyzed by electrochemical analytical instrument CHI-6122E (commercially available from CH instruments) to measure the cyclic voltammetry (CV) spectrum of the electrochromic composition (scan rate was 50 mV/s). The CV spectrum of the electrochromic composition is shown in FIG. 1D.

In addition, different driving voltages were applied to the electrochromic composition to measure its absorption spectra, as shown in FIG. 2D. As known from FIG. 2D, when the driving voltage achieved 0.9 V, the electrochromic composition would have an electrochromic effect. When no driving voltage was applied, the electrochromic composition was colorless (L*=97.90, a*=1.08, b*=2.68). When a driving voltage of 0.9 V was applied, the electrochromic composition changed to cobalt blue (L*=55.69, a*=−8.92, b*=−46.47). When a driving voltage of 1.8 V was applied, the electrochromic composition changed to deep blue (L*=42.90, a*=−12.80, b*=−45.93). The driving voltages and the color variation effect of the electrochromic composition are tabulated in Table 1.

0.5 μmole of TPA-3N was dissolved in 1 mL of γ-butyrolactone solution of TBAP (1M), and analyzed by a three-electrode system to measure the CV spectrum of TPA-3N, as shown in FIG. 3D. The CV spectra of the TPA-3N solution after different scan cycles (scan rate was 100 mV/s) are shown in FIG. 4D. The oxidation-reduction potentials of TPA-3N could be known from the CV spectra. The absorbance residual value (of maximum UV absorption band) after 10000 cycles from 0.5 V to −0.1 V is tabulated in Table 2.

### Comparative Example 1

An electrochromic composition was prepared, which contained solvent of γ-butyrolactone (0.048 mL), 0.72 μmole of the triphenyl amine compound 3PiAB (0.015 M), 0.72 μmole of the heptyl viologen HV (0.015 M), and 4.8 μmole of TBABF4 (0.1 M). The electrochromic composition was interposed between two ITO glasses (2 cm*2 cm), and analyzed by electrochemical analytical instrument CHI-6122E (commercially available from CH instruments) to measure the cyclic voltammetry (CV) spectrum of the electrochromic composition (scan rate was 50 mV/s). The CV spectrum of the electrochromic composition is shown in FIG. 1E. In addition, the color variation effect of the electrochromic composition under a driving voltage of 1.2 V was only about 14.2%. The driving voltage and the color variation effect of the electrochromic composition are tabulated in Table 1.

0.5 μmole of 3PiAB was dissolved in 1 mL of γ-butyrolactone solution of TBAP (1M), and analyzed by a three-electrode system to measure the CV spectrum of 3PiAB, as shown in FIG. 3E. The oxidation-reduction potentials of 3PiAB could be known from the CV spectrum.

TABLE 1

| | Electrochromic composition | | | Test result | | |
|---|---|---|---|---|---|---|
| | Oxidizable | Reducible | | Driving | L* before | L* after |
| | material | material | Electrolyte | voltage | driving | driving |
| Example 1 | TPA-ADM | HV | TBABF4 | 0.9 V | 97.87 | 53.50 |
| Example 2 | TPA-1N | | | 1.2 V | 97.52 | 47.9 |
| Example 3 | TPA-2N | | | 0.9 V | 98.23 | 69.29 |
| Example 4 | TPA-3N | | | 0.9 V | 97.90 | 55.69 |
| Comparative | 3PiAB | | | 0.9 V | 88.93 | 88.93 |
| Example 1 | | | | 1.2 V | 88.93 | 74.79 |

TABLE 2

| | | | | | | Absorption residual |
|---|---|---|---|---|---|---|
| | | First oxidation- | | Second oxidation- | | value (of maximum |
| | | reduction potential | | reduction potential | | UV absorption |
| | Oxidizable | Oxidation | Reduction | Oxidation | Reduction | band) after 10000 |
| | material | (V) | (V) | (V) | (V) | cycles |
| Example 1 | TPA-ADM | 0.38 | 0.28 | 0.73 | 0.63 | 88% |
| Example 2 | TPA-1N | 0.52 | 0.42 | 0.98 | 0.85 | 92% |
| Example 3 | TPA-2N | 0.43 | 0.3 | 0.79 | 0.65 | 90% |
| Example 4 | TPA-3N | 0.34 | 0.18 | 0.73 | 0.57 | 90% |
| Comparative | 3PiAB | 0.80 | 0.56 | 1.3 | 1.3 | (No color variation |
| Example 1 | | | | | | effect when the |
| | | | | | | driving voltage was |
| | | | | | | 0.5 V to −0.1 V) |

As known from Table 1, the electrochromic compositions in Examples had low driving voltages (e.g. 0.9 V to 1.2 V) and high contrasts (e.g. differences of L* were large).

The heptyl viologen in Examples above had a first reduction potential of 0.45 V and a second reduction potential of 0.85 V. As shown in Table 2, the oxidizable compounds in Examples had first oxidation potentials to match the first reduction potential of the heptyl viologen (e.g. differences therebetween were less than 0.15 V) and second oxidation potentials to match the second reduction potential of the heptyl viologen (e.g. differences therebetween were less than 0.15 V), thereby improving the oxidation-reduction cycling stability and lowering the driving voltage.

As known from the above Examples and the Comparative Example, not all the triphenyl amine compounds were suitable to collocate with the heptyl viologen to serve as the electrochromic composition. When some triphenyl amine compound (e.g. 3PiAB in Comparative Example 1) was collocated with the heptyl viologen, the electrochromic driving voltage will be obviously overly high (>1.2 V) and could not achieve the low driving voltage (e.g. 0.9 V to 1.2 V) of the electrochromic compositions in Examples. In addition, the electrochromic composition containing some triphenyl amine molecule (e.g. 3PiAB in Comparative example 1) had low contrast (i.e. the difference of L* before and after driving was small).

The oxidizable compound could be used in the electrochromic composition. The electrochromic device containing the electrochromic composition had a decreased driving voltage, an enhanced contrast before and after driving, and a cycling stability.

The electrochromic device could have a lower driving voltage, an enhanced contrast before and after driving, and a cycling stability through the electrochromic composition of the disclosure.

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. An oxidizable cc having a chemical structure of 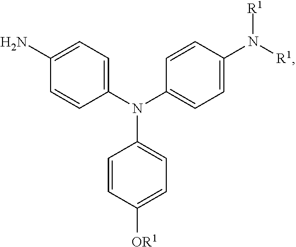
wherein each R¹ is independently C₁₋₃ alkyl group.

2. The oxidizable compound as claimed in claim 1, wherein the oxidizable compound has a chemical structure of

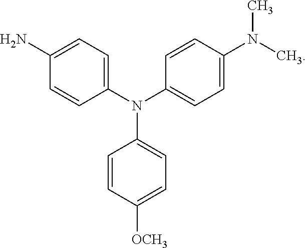 [SMILES: COc1ccc(N(c2ccc(N)cc2)c2ccc(N(C)C)cc2)cc1]

3. An electrochromic composition, comprising:
an oxidizable compound;
a reducible compound; and
a solvent,
wherein the oxidizable compound includes 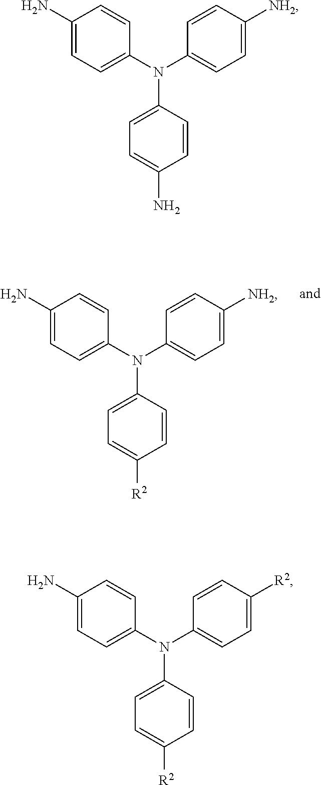
wherein each R² is independently —N(R¹)₂, —OR¹, or —R¹, and R¹ is C₁₋₃ alkyl group;
wherein the reducible compound has a chemical structure of 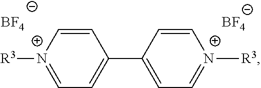
each R³ is independently C₄₋₁₂ alkyl group, —OCₙH₂ₙ₊₁, 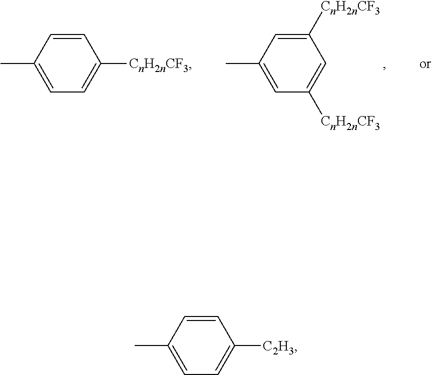
and n=0˜7.

4. The electrochromic composition as claimed in claim 3, wherein the oxidizable compound has a chemical structure of

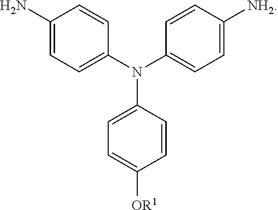

5. The electrochromic composition as claimed in claim 3, wherein the oxidizable compound has a chemical structure of

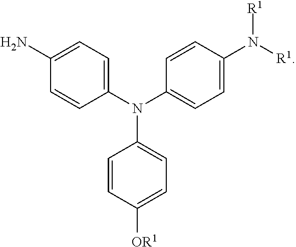

6. The electrochromic composition as claimed in claim 3, wherein the oxidizable compound has a chemical structure of

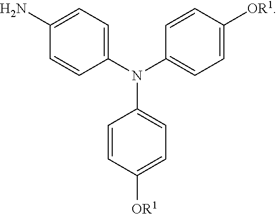

7. The electrochromic composition as claimed in claim 3, wherein the solvent comprises propylene carbonate, γ-butyrolactone, acetonitrile, propionitrile, glutaronitrile, methylglutaronitrile, 3,3′-oxydipropionitrile, hydroxypropionitrile, dimethylformamide, N-methyl tetrahydropyrrolidone, sulfolane, 3-methylsulfolane, or a combination thereof.

8. The electrochromic composition as claimed in claim 3, further comprising
an electrolyte.

9. An electrochromic device, comprising:
a first transparent conductive layer;
a second transparent conductive layer; and
the electrochromic composition as claimed in claim 3 disposed between the first transparent conductive layer and the second transparent conductive layer.

10. The electrochromic device as claimed in claim 9, wherein the first transparent conductive layer and the second transparent conductive layer comprise indium tin oxide, indium zinc oxide, aluminum zinc oxide, cadmium tin oxide, tin oxide, or zinc oxide.
